(19) United States
(12) Patent Application Publication
(10) Pub. No.: US 2012/0012121 A1
(43) Pub. Date: Jan. 19, 2012

(54) METHOD OF FOLLICULAR MUSCLE AND NERVE STIMULATION

(76) Inventors: Deanna L. Sipe, Indianapolis, IN (US); Thomas Meyer, Indianapolis, IN (US)

(21) Appl. No.: 12/837,673

(22) Filed: Jul. 16, 2010

(51) Int. Cl. A61B 17/00

(52) U.S. Cl. 128/898

(57) ABSTRACT

A method of follicular muscle and nerve stimulation configured to generate a follicular stimulation effect in high contrast to epidermal stimulation. Steps include performing a plurality of successive longitudinal hair pulls, transverse cascading hair pulls, cascading nape pulls, performing a series of alternating transverse cascading lateral-to-crown pulls; crown pulls; twisted temple-to-temple pulls; and measuring a physiological characteristic of an individual receiving follicular nerve and muscle stimulation using a measuring device. Hair pulls include collecting a set of hair strands, thereby forming a hair strand assemblage, and pulling the hair strand assemblage. Pulls are directed longitudinally and/or transversely. Cascading pulls include releasing portions of the hair strand assemblage in a cascading manner as the hair is pulled.

### BACKGROUND OF THE INVENTION

1. Field of the Invention

The present invention relates to stimulation methods, specifically a method of follicular muscle and nerve stimulation.

2. Description of the Related Art

Techniques have been developed in reference to stimulating or providing treatment or therapy to portions of the human body. These include massage techniques, chiropractic techniques, sports medicine, yoga, physical therapy, stretching techniques and the like. It is generally understood that improvements in health and well being may be derived from some kinds of physical stimulation of body tissue, while other kinds of stimulation may be harmful. Accordingly, improvements in such techniques are constantly being made in order to promote higher and higher levels of health and well-being for recipients of the techniques. Some improvements have been made in the field. Examples of such improvements are described below in their own wording, and the supporting teachings of each reference are incorporated by reference herein:

U.S. Pat. No. 7,399,286, issued to Jackson, discloses a multi-layered massage devices for providing a pleasurable massage sensation; for simulating a lubricant-aided massage on the skin of an animal, without the use of a lubricant; for treating or preventing a cause or a symptom of a disease, disorder, or condition in an animal in need thereof, by administering a therapeutic massage; for genital stimulation for the pleasure of a male or female human; and for producing a low friction interface between a surface of a human body and a surface of another object.

U.S. Pat. No. 6,911,012, issued to Kahn, discloses a massage device for manipulating a user includes a support member and a manipulator carried by the support member. The manipulator is adapted to engage and apply a shear force to the user. The manipulator is rotatable about at least one axis relative to the support member and a brake operably connected to the manipulator selectively adjusts rotation of the manipulator about the at least one axis so that the shear force applied to the user by the manipulator is adjusted. In automatically controlled embodiments, a controller adjusts the braking force according to signals received from a detector to prevent or reduce slippage of the roller and to dynamically control the magnitude of the shear force applied to the user. In manually embodiments, an operator adjusts the braking force to a fixed magnitude or dynamically adjusts the braking force to control the shear force applied to the user.

U.S. Pat. No. 6,174,298, issued to Quinn et al., discloses a massaging element for delivering a long stroke massage, the massaging element having at least one pair of symmetrical arc-shaped spring elements extending from a central driving component and having replaceable massaging units such as rollers at their free ends. A driving unit delivers reciprocating motions to the central driving component causing compressing and outward movements of the massaging units. A power or manually driven massager incorporates a plurality of the massaging elements for a wide area massage of the patient's body. The massager is easily foldable for storage or transport.

U.S. Pat. No. 5,840,048, issued to Cheng, discloses a method of treating the skin by specific massage procedure, using one or more brushes. The method comprises the steps of providing a facial and a body massaging brush. A clockwise brush massage is applied to stimulate specific designated areas of the body for a predetermined period of time. The brush massage is performed in a specific sequence of the designated areas in compliance with a network of acupuncture points. The method of this invention improves blood circulation and simultaneously provides an exfoliation of skin tissue.

U.S. Pat. No. 5,707,346, issued to Graston, discloses a method and a system for use by a trainer in performing soft tissue massage. The system includes a tool including a handle portion and a skin engagement portion that is configured to generally match the contour of the impaired area of soft tissue to be treated. The tool is connected to a source of electrical current for providing electrical stimulation to the impaired area during treatment with the tool. The skin engagement portion noninvasively engages the skin to allow the user to locate fibrous adhesions that are attached to the underlying soft tissue areas.

The inventions heretofore known suffer from a number of disadvantages which include being limited in application, being limited in use, being difficult to perform, being unduly complex, being ineffective, being expensive, being time consuming, and/or being inefficient.

The human head includes a great variety of organs and structures. One very important organ is the skin. The skin is further composed of a great variety of structures and systems that operate to protect the inner organs from germs, disease, trauma, temperature, and the like. The skin includes an epidermal layer (outer layer), called the epidermis, that protects a dermal layer (inner), called the dermis, disposed over a subdermal layer, called the subcutis. Hairs extend from the dermis through the epidermis to the exterior of the body and perform a variety of protective and decorative functions. In humans and among most human cultures, hair has taken on decoration as a primary role. In most mammals, the follicles play a powerful role in survival of the individual.

Hair follicles, in a larger sense of the term, are systems of interoperating components located in close proximity within the skin. One may consider the specific follicles to be central units about which many other components are arranged, thereby providing benefit and function to the skin.

Of note, there is a set of muscles known as the arrectores pilorum (singular: arrector pili). The arrectores pilorum are very small muscles that attach to the hair follicles in the dermal layer of the skin. These muscles are not under conscious control, but instead are enervated by the sympathetic branch of the autonomic nervous system. They are generally activated in response to cold, fear, and other stimulation. When activated, the angular position of the hair follicle is shifted, resulting in what are generally known as “goose bumps.”

A set of nerve fibers, called dermal nerves, extend through the dermis and connect to and about hair follicles. These nerve fibers send and receive information to and from the hair follicles and surrounding systems (sweat glands, etc.) to facilitate the proper functioning of the systems and inform the brain of local issues. Different nerve fibers report/send different information based on the orientation, termination locations, and receptor configurations of the fibers. Different fibers and/or fiber portions are generally very specialized, providing very specific information about very local events.

Other components, such as but not limited to the sebaceous glands, are positioned and oriented to provide beneficial functions in association with hair follicles. Partial or complete failure by these systems results in skin issues, such as but not limited to sores, dryness, pustules, and the like.

What is needed is a method of follicular muscle and nerve stimulation that solves one or more of the problems described herein and/or one or more problems that may come to the attention of one skilled in the art upon becoming familiar with this specification.

### SUMMARY OF THE INVENTION

The present invention has been developed in response to the present state of the art, and in particular, in response to the problems and needs in the art that have not yet been fully solved by currently available methods of stimulation. Accordingly, the present invention has been developed to provide a method of follicular muscle and nerve stimulation.

According to one embodiment of the invention, there is a method of follicular muscle and nerve stimulation that may be configured to generate a follicular stimulation effect in high contrast to epidermal stimulation. The method may include: performing a plurality of successive longitudinal hair pulls, wherein each of the longitudinal hair pulls may include collecting a set of hair strands, thereby forming a hair strand assemblage, and/or pulling the hair strand assemblage in a direction that may be substantially aligned to the longitudinal orientation of the hair strand assemblage; and/or performing a plurality of successive transverse cascading hair pulls, wherein each of the transverse cascading hair pulls may include collecting a set of hair strands, thereby forming a hair strand assemblage, and/or pulling the hair strand assemblage in a direction that may be substantially transverse to the longitudinal orientation of the hair strand assemblage, and/or releasing portions of the hair strand assemblage in a cascading manner as the hair is pulled.

The method may include performing a series of transverse cascading nape pulls; alternating transverse cascading lateral-to-crown pulls; transverse cascading crown pulls; longitudinal nape pulls; transverse cascading nape to crown pulls; crown pulls; twisted temple-to-temple pulls; crown-to-nape pulls.

The method may include measuring a physiological characteristic of an individual receiving follicular nerve and muscle stimulation.

Moreover, the method may include wherein a first step is performed before performance of a second step. The method may also include wherein the first step is repeated a plurality of times using a plurality of differing pulls, a plurality of times, and then the second step is subsequently performed a plurality of times. The method may include performing a series of low stimulation pulls then performing a series of high stimulation pulls. Low stimulation pulls may include pulls wherein a large quantity of hairs is included in a hair assemblage and/or longitudinal pulls. High stimulation pulls may include pulls wherein a small quantity of hairs is included in a hair assemblage and/or transverse and/or cascading pulls.

Reference throughout this specification to features, advantages, or similar language does not imply that all of the features and advantages that may be realized with the present invention should be or are in any single embodiment of the invention. Rather, language referring to the features and advantages is understood to mean that a specific feature, advantage, or characteristic described in connection with an embodiment is included in at least one embodiment of the present invention. Thus, discussion of the features and advantages, and similar language, throughout this specification may, but do not necessarily, refer to the same embodiment.

Furthermore, the described features, advantages, and characteristics of the invention may be combined in any suitable manner in one or more embodiments. One skilled in the relevant art will recognize that the invention can be practiced without one or more of the specific features or advantages of a particular embodiment. In other instances, additional features and advantages may be recognized in certain embodiments that may not be present in all embodiments of the invention.

These features and advantages of the present invention will become more fully apparent from the following description and appended claims, or may be learned by the practice of the invention as set forth hereinafter.

### BRIEF DESCRIPTION OF THE DRAWINGS

In order for the advantages of the invention to be readily understood, a more particular description of the invention briefly described above will be rendered by reference to specific embodiments that are illustrated in the appended drawing(s). It is noted that the drawings of the invention are not to scale. The drawings are mere schematics representations, not intended to portray specific parameters of the invention. Understanding that these drawing(s) depict only typical embodiments of the invention and are not, therefore, to be considered to be limiting its scope, the invention will be described and explained with additional specificity and detail through the use of the accompanying drawing(s), in which:

FIG. 1 is a back perspective view of performance of a longitudinal nape pull using an implement, according to one embodiment of the invention;

FIG. 2 is a back perspective view of performance of a transverse cascading nape pull, according to one embodiment of the invention;

FIG. 3 is a back perspective view of performance of a transverse cascading crown pull, according to one embodiment of the invention;

FIG. 4 is a back perspective view of performance of a longitudinal nape pull, according to one embodiment of the invention;

FIG. 5 is a back perspective view performance of alternating transverse cascading lateral-to-crown pulls, according to one embodiment of the invention;

FIG. 6 is a back perspective view of performance of longitudinal hair pulls, according to one embodiment of the invention;

FIG. 7 is a back perspective view performance of a transverse cascading nape pull, according to one embodiment of the invention;

FIG. 8 is a top plan view of performance of a twisted temple-to-temple pull, according to one embodiment of the invention;

FIG. 9 is a side perspective view performance of a crown-to-nape pull, according to one embodiment of the invention;

FIG. 10 is a back perspective view of performance of a transverse cascading nape-to-crown pull, according to one embodiment of the invention;

FIG. 11 is a back perspective view of a longitudinal hair pull, according to one embodiment of the invention;

FIG. 12 is a back perspective view of performance of a nape-to-crown pull, according to one embodiment of the invention;

FIG. 13 is a back perspective view of nape-to-crown pull, according to one embodiment of the invention; and

FIG. 14 is a back perspective view of a matched pair of transverse lateral-to-crown pulls, according to one embodiment of the invention.

### DETAILED DESCRIPTION OF THE INVENTION

For the purposes of promoting an understanding of the principles of the invention, reference will now be made to the exemplary embodiments illustrated in the drawing(s), and specific language will be used to describe the same. It will nevertheless be understood that no limitation of the scope of the invention is thereby intended. Any alterations and further modifications of the inventive features illustrated herein, and any additional applications of the principles of the invention as illustrated herein, which would occur to one skilled in the relevant art and having possession of this disclosure, are to be considered within the scope of the invention.

Reference throughout this specification to an “embodiment,” an “example” or similar language means that a particular feature, structure, characteristic, or combinations thereof described in connection with the embodiment is included in at least one embodiment of the present invention. Thus, appearances of the phrases an “embodiment,” an “example,” and similar language throughout this specification may, but do not necessarily, all refer to the same embodiment, to different embodiments, or to one or more of the figures. Additionally, reference to the wording “embodiment,” “example” or the like, for two or more features, elements, etc. does not mean that the features are necessarily related, dissimilar, the same, etc.

Each statement of an embodiment, or example, is to be considered independent of any other statement of an embodiment despite any use of similar or identical language characterizing each embodiment. Therefore, where one embodiment is identified as “another embodiment,” the identified embodiment is independent of any other embodiments characterized by the language “another embodiment.” The features, functions, and the like described herein are considered to be able to be combined in whole or in part one with another as the claims and/or art may direct, either directly or indirectly, implicitly or explicitly.

As used herein, “comprising,” “including,” “containing,” “is,” “are,” “characterized by,” and grammatical equivalents thereof are inclusive or open-ended terms that do not exclude additional unrecited elements or method steps. “Comprising” is to be interpreted as including the more restrictive terms “consisting of” and “consisting essentially of.”

As used herein, a pull characterized as “longitudinal” is one wherein the direction of the pull is substantially aligned with the length of the associated hair assemblage.

As used herein, a pull characterized as “transverse” is one wherein the direction of the pull is substantially aligned perpendicular to with the length of the associated hair assemblage.

As used herein, a pull characterized in association with a portion of the head, including but not limited to “nape,” “top,” “temple,” and the like is a pull wherein a portion of the pull occurs near that portion of the head.

As used herein, a pull characterized as “cascading” in one wherein hair is released over time from the pull during the pull. Generally, when performing a cascading pull, the practitioner will utilize a tool or hand configuration that permits hair to escape control of the tool or hand along the patch of the pull.

As used herein, a pull characterized as “alternating” is one wherein the pull is performed by each hand in a repeating pattern wherein a hand is, generally speaking, beginning a pull as another hand is, generally speaking, finishing a similar pull. Exact timing is not necessary but may be advantageous.

As used herein, a pull characterized as “twisted” is one wherein force is applied to a hair assemblage that causes the hair assemblage to rotate about the long axis of the hair assemblage.

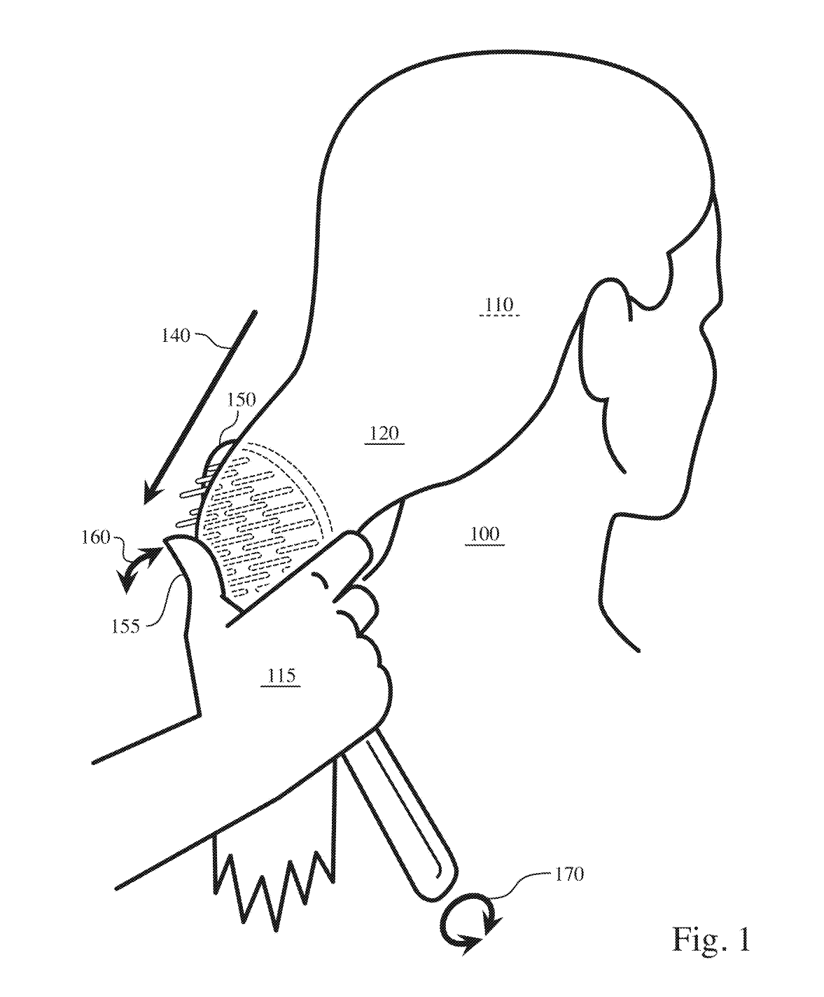

FIG. 1 is a back perspective view of performance of a longitudinal nape pull using an implement, according to one embodiment of the invention. There is shown an implement 150 (hair brush, brush head) held in the hand 115 of a practitioner. The illustrated implement 150 is pressed into a hair assemblage 120 from an underside of the illustrated assemblage 120. The hand 115 of the practitioner includes a thumb 155 that is applied against the hair assemblage 120 and towards the implement 150. Accordingly, by controlling the position 160 and force of the thumb in relation to the hair assemblage and implement 150, the practitioner may control the friction or drag during the hair pull.

This technique may be referred to as a “thumb-in” technique. In the illustrated “thumb-in” technique, the thumb 155 and the brush head 150 are used to pick up or collect the hair assemblage 120 therebetween. The brush head 150 is placed into the hair 120, below a nape region 110, and the thumb 155 pushes the hair 120 into the bristles of the brush head 150, thereby creating drag. The hand 115 and the brush head 150 is pulled away from a neck region 100 in a direction 140 that is substantially aligned with the longitudinal axis of the hair of the hair assemblage 120. The following non-limiting examples of techniques/variations may be utilized to alter the experience: slow pull back of the brush and thumb; and/or a twist 170 to the angle of the brush head to create additional drag.

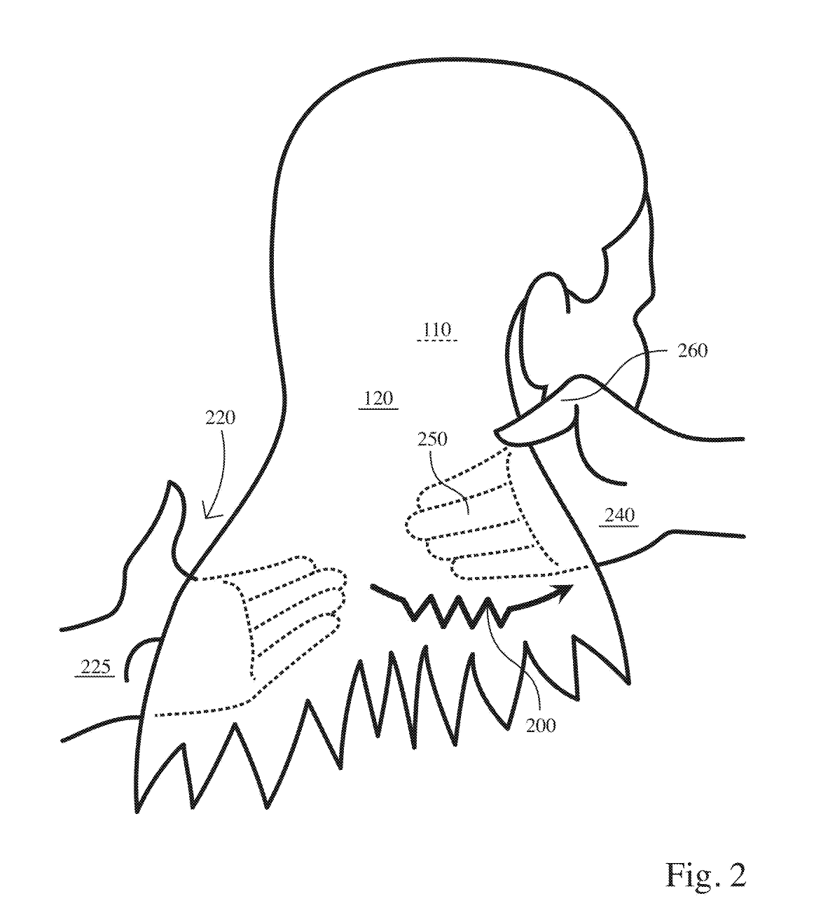

FIG. 2 is a back perspective view of performance of a transverse cascading nape pull, according to one embodiment of the invention. There is shown a left hand 225 and a right hand 240 of a practitioner, wherein the right hand 240 is in a flat-duck handed configuration about a hair assemblage 120 with the fingers 250 aligned and substantially straightened and the thumb 260 of the right hand pressed thereagainst. The right hand 240 is oriented such that the fingers 250 are substantially perpendicular to the longitudinal orientation of the hair assemblage. The hair assemblage is formed at the back of the neck 100 near the nape 110. The left hand 225 is in an open configuration 220 set to receive cascading hair from the hair assemblage 120.

The illustrated technique may be referred to as a “back-tail drag” and is configured to provide follicular muscle and nerve stimulation. In operation, an assemblage of hair 120 is collected into a pony tail with a left hand 225 and a right hand 240. The right hand 240 is configured in a flat-duck handed technique. Using the flat-duck handed technique, a practitioner is able to regulate a pressure applied to the hair assemblage 120. The hand 240 is pulled 200 sideways, or in other words, substantially perpendicular to the longitudinal axis of the hair assemblage 120. The hair assemblage 120 progressively falls from the grip of the right hand, thereby cascading out of one hand 240 and into the other hand 225. Catching the hair in the left hand 225 permits the practitioner to begin collection of a new hair assemblage to be pulled by the left hand 225. Accordingly, the process may be repeated over and over, alternating direction and which hand is pulling.

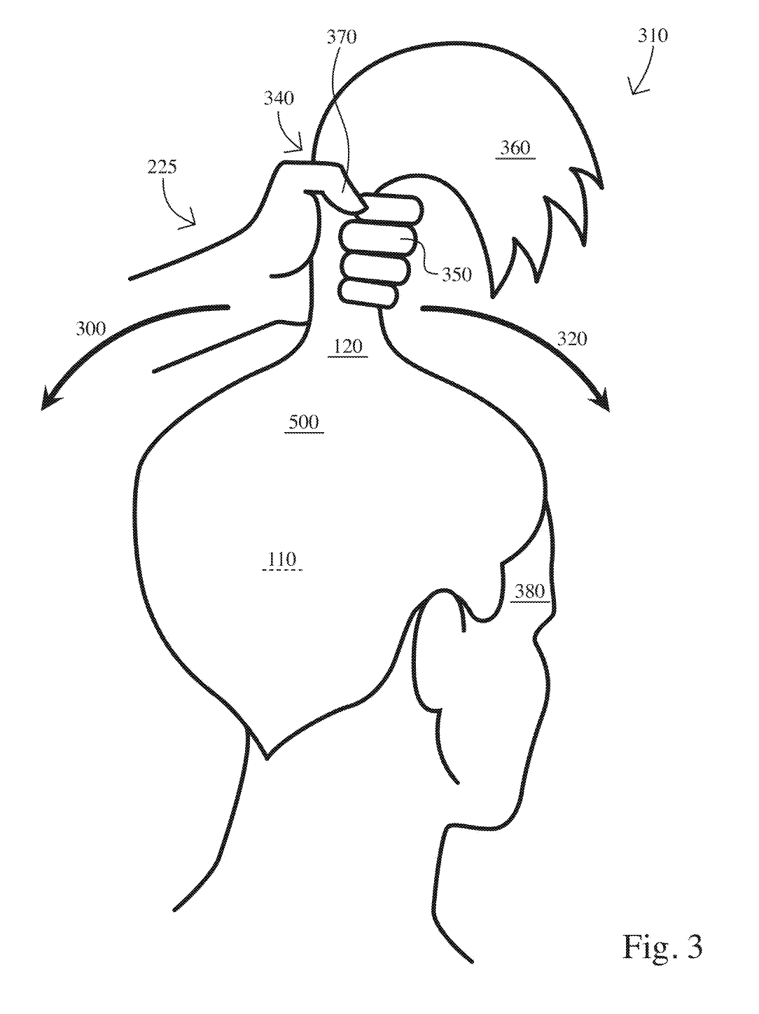

FIG. 3 is a back perspective view of performance of a transverse cascading crown pull, according to one embodiment of the invention. There is shown a left hand 225 of a practitioner, wherein the left hand 225 is in a four finger hand configuration 340 about a hair assemblage 120 with the fingers 350 aligned and substantially closed against a palm/thumb 370. The left hand 225 is oriented such that the fingers 350 are substantially perpendicular to the longitudinal orientation of the hair assemblage 120. The four finger hand configuration 340 forms a top pony tail 360 extending from the left hand 225.

The illustrated technique may be referred to as a “top tail drag” and is configured to provide follicular muscle and nerve stimulation. In operation, an assemblage of hair 120 is collected into a top pony tail 360 with a left hand 225. The left hand 225 is configured in a four finger hand configuration The top tail drag 310 includes a four finger hand technique 340 pressing against a palm/thumb base 370. The top pony tail 360, is collected from a crown region 500 of the head, is pulled to a left side 300 or a right side 320 in succession, thereby allowing the assemblage of hair 120 to slide along the left hand 225. The assemblage of hair 120 in the top pony tail 360 does not fall through unless the hair 120 is too short. The assemblage of hair 120 may stay in the left hand 225 or fall out. The top tail drag 310 includes changing directions 300, 320 when the hands alternate in series. The left hand 225 pulls to the left 300 and the right hand may pull to the right 320. The tension is regulated by the fingers 350 and the palm/thumb base 370. The palm/thumb base 370 of the hand 225 will be substantially faced up when the top tail drag 310 is complete. The top tail drag 310 also includes alternating the left hand and the right hand by scooping up the assemblage of hair 120 and pulling back across to the other side. The top tail drag 310 stimulates a forehead region 380 and a nape region 110 of the head.

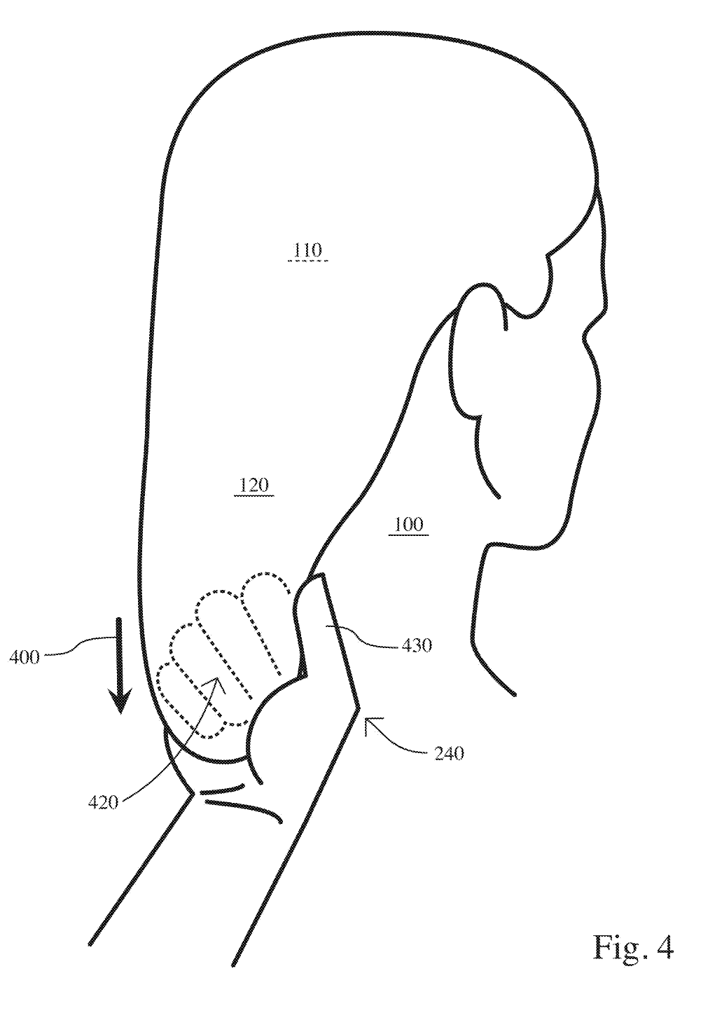

FIG. 4 is a back perspective view of performance of a longitudinal nape pull, according to one embodiment of the invention. There is shown a right hand 240 of a practitioner, wherein the right hand 240 is in a closed hand configuration about a hair assemblage 120 with the fingers 420 aligned and substantially closed against the thumb 430. The right hand 240 is orientated such that the fingers 420 are substantially diagonal to the longitudinal orientation of the hair assemblage 120. The hair assemblage is formed at the back of the neck, below the nape 110.

The illustrated technique includes a series of longitudinal nape pulls including grabbing the ends of the hair, and forming an assemblage of hair 120 after a nape region 110 and pulling the hair 120 downward 400. As illustrated, the hair assemblage 120 is configured in a pony tail configuration. The longitudinal nape pull includes rolling the hair ends into a hand 240 between a thumb 430 and fingers 420. Nape pulls also includes pulling from side to side or as illustrated in FIG. 4, pulling the hair 120 straight downward 400 away from a neck region 100.

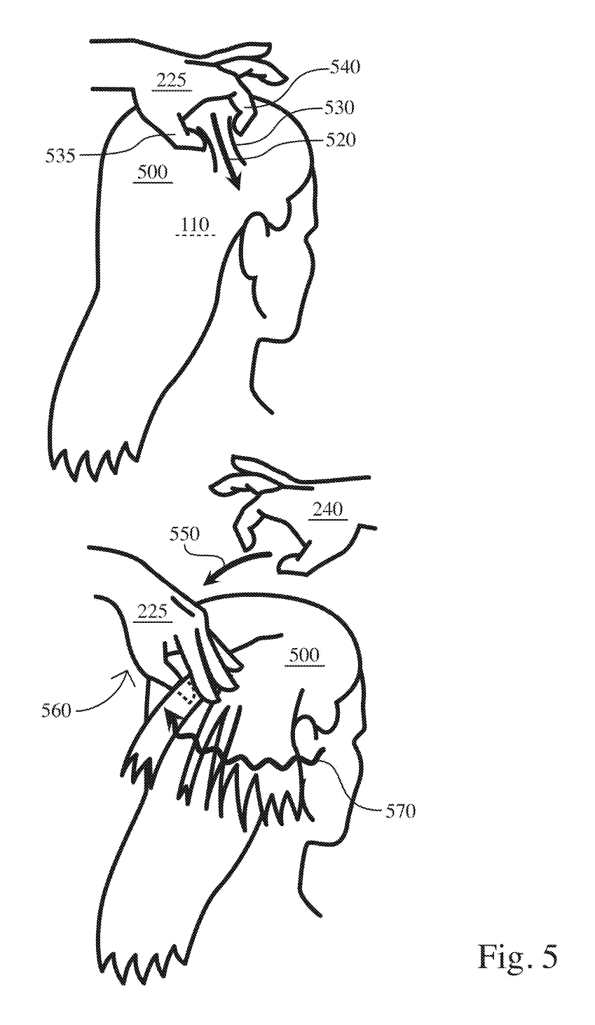

FIG. 5 is a back perspective view performance of alternating transverse cascading lateral-to-crown pulls, according to one embodiment of the invention. There is shown a left hand 225 and a right hand 240 of a practitioner, wherein the left hand 225 is in a pinching hand configuration about a hair assemblage, with the thumb 535 and the forefinger 540 pinching the hair assemblage therebetween during the pull. The right hand 240 is also in a pinching hand configuration, poised to pinch an assemblage of hair after gathering an assemblage of hair. The hair assemblage is formed about a crown region 500 of the head, above a nape region 110.

The illustrated technique may be referred to as a “criss-cross cascade” and is configured to provide follicular muscle and nerve stimulation. In operation, an assemblage of hair is collected by the thumb and forefinger of a hand of a practitioner. The left hand and the right hand are configured in a pinching hand configuration on a crown region of the head, above a nape region. The criss-cross cascade technique 570 includes dividing an assemblage of hair down opposite sides about a crown region; and the practitioner uses each hand to create a part 530, with a thumb 535 and forefinger 540. The thumb 535 and forefinger 540 pinches together 530 and pulls the hair back upwards 520, and allowing the assemblage of hair to slip out between the thumb 535 and forefinger 540 in a flat-ducked configuration 560. The assemblage of hair is slightly pulled across the neck, thereby creating tension as the hair slips out of the thumb 535 and fingers. The technique may include repeating the criss-cross cascade technique 570 with each hand 225, 240 and/or repeating on generally opposite sides of the crown region of the assemblage of hair 550.

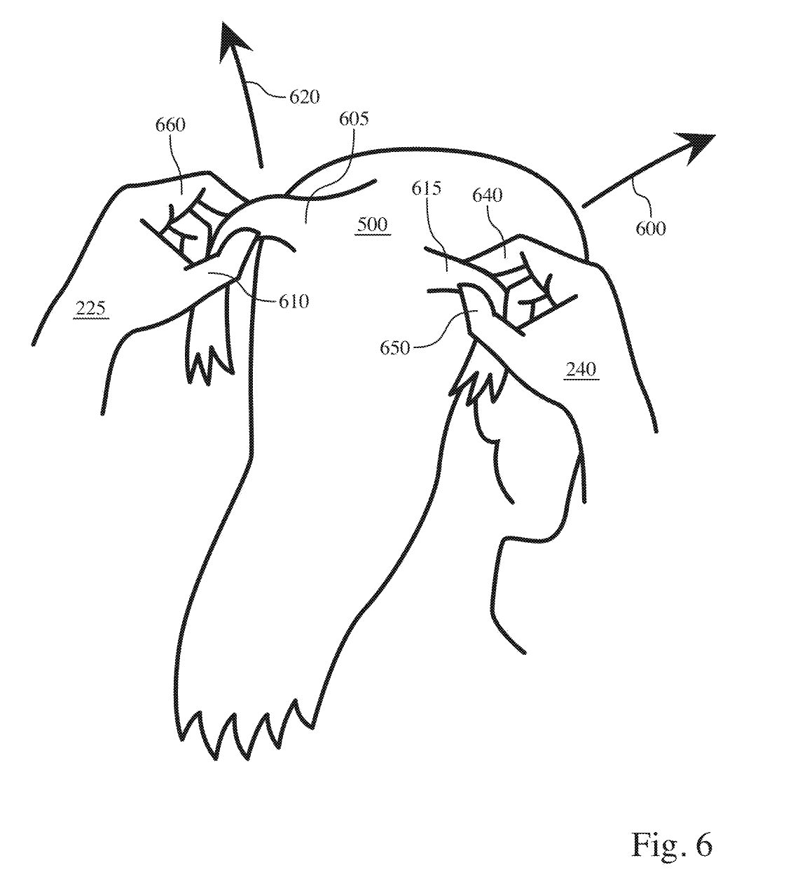

FIG. 6 is a back perspective view of performance of longitudinal hair pulls, according to one embodiment of the invention. There is shown a left hand 225 and a right hand 240 of a practitioner; wherein a thumb 610, 650 and a forefinger 660, 640 of each hand collects an assemblage of hair 605, 615. The assemblage of hair 605, 615 is formed at the top of a crown region. The left hand and the right hand are oriented in a “pinching” technique to collect the assemblage of hair.

The illustrated technique may be referred to as a “pinching” technique and is configured to provide follicular muscle and nerve stimulation. In operation, a practitioner forms a hair strand assemblage 605, 615 with a thumb and forefinger. The hair strand assemblage 605 is pulled in a direction substantially aligned to the longitudinal orientation 620 of the hair strand assemblage 605. As illustrated, the longitudinal hair pulls 600, 620 includes pinching a hair strand assemblage 605, 615, of a crown region 500 of the head, and pulling the hair 605, 615 away from the head. The hair swatches between a thumb 610, 650 and a forefinger 660, 640, of each hand 225, 240 and pulls the hair assemblage 605, 615 outward a couple of inches and putting it back down. The longitudinal hair pulls includes performing the hair pulls in random spots or in a pattern over the head. The method may include alternating the right hand 240 and the left hand 225, so that the experience is continuous and successive. The method may also include pulling the hair in a substantially outward direction 600.

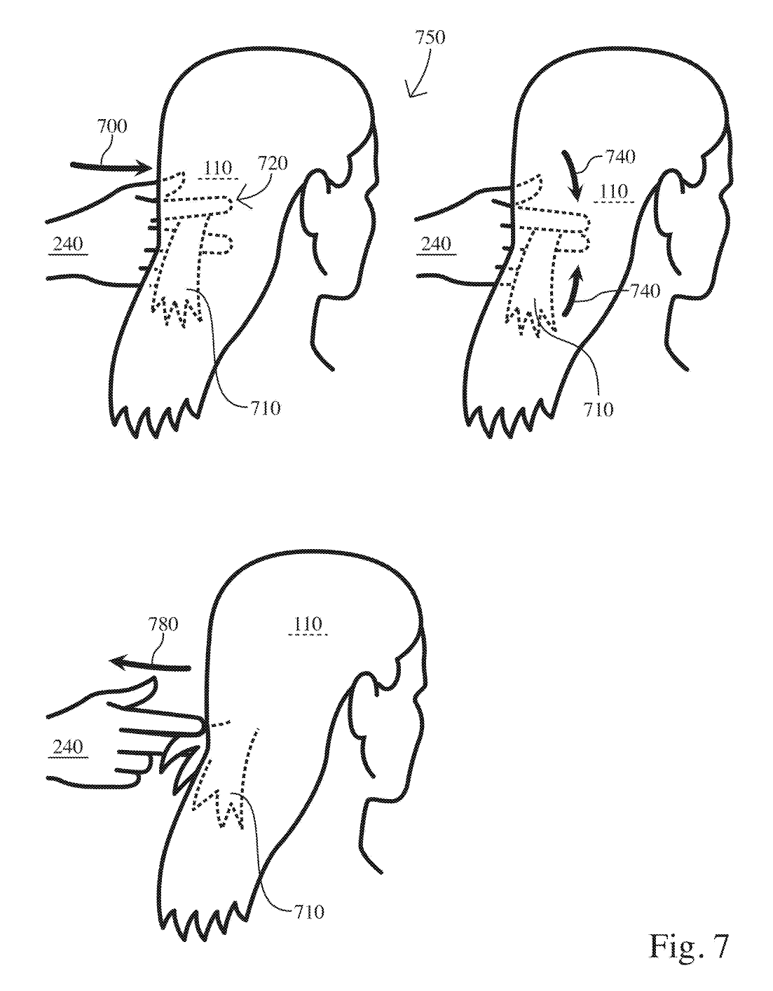

FIG. 7 is a back perspective view performance of a transverse cascading nape pull, according to one embodiment of the invention. There is shown a right hand 240 of a practitioner; wherein the right hand 240 is configured in a “scissor” technique. The practitioner collects an assemblage of hair, under a nape region, with a third finger and a forefinger. The fingers are substantially perpendicular to the longitudinal orientation of the hair assemblage.

The illustrated technique may be referred to as an “undercover scissors” technique 750; wherein a third and a forefinger reaches under a nape region 110 of the head 700. In operation, the fingers are spreaded out to gather an assemblage of hair 710 in a scissor-like configuration 720. The assemblage of hair 710 is trapped between the fingers 740 and pulled sideways, across a neck region, allowing the hair to slip out of the fingers 780. The undercover scissors technique 750 preferred embodiment is performed when someone doesn't want to mess up the hair or when someone is out in public. The undercover scissors technique 750 may use 2, 3, 4, 5 fingers in performing the series of transverse cascading nape pulls, trapping hair assemblages between adjacent fingers. The undercover scissors technique may include performing multiple undercover scissors technique pulls simultaneously.

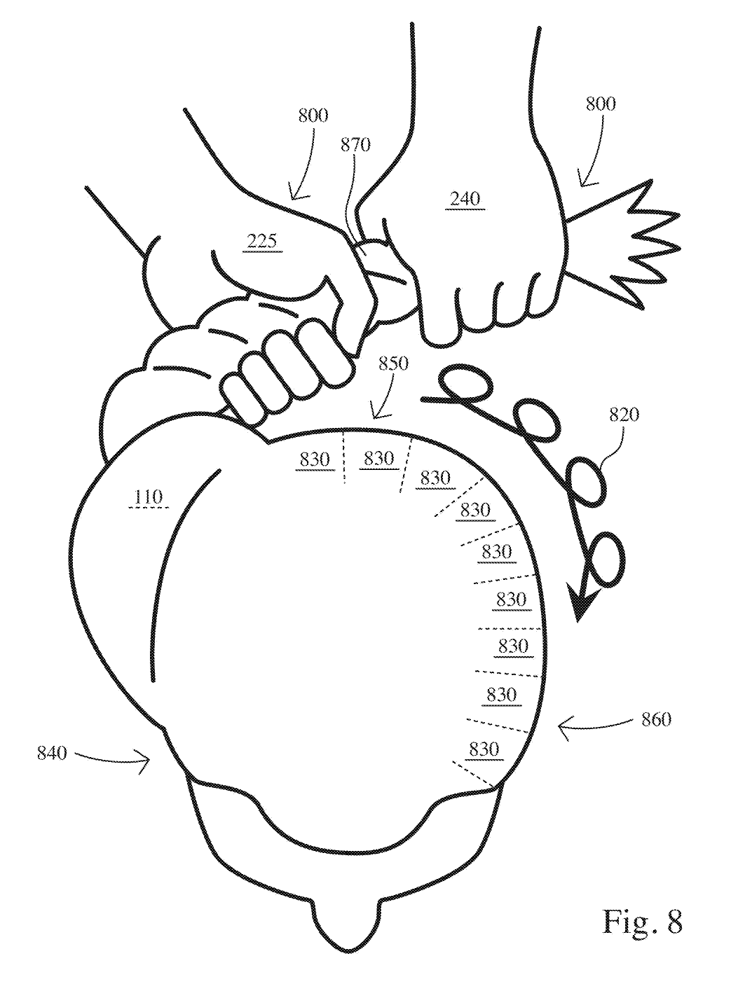

FIG. 8 is a top plan view of performance of a twisted temple-to-temple pull, according to one embodiment of the invention. There is shown a left hand 225 and a right hand 240; wherein the left and right hands are grasping an assemblage of hair 800. The assemblage of hair is grasped by the fingers and thumbs of each hand. The assemblage of hair is twisted substantially behind the head 850 around a nape region 110. The assemblage of hair is held within the each hand, with the fingers and thumb applying pressure to the palm of the hand, thereby securing the assemblage between both hands.

The illustrated technique may be referred to as a “twisted temple-to-temple pull” 820. In operation, the twisted temple-to-temple pull 820 includes a left hand 225 and a right hand 240 using a four finger hand configuration pressing against a palm/thumb base 800, thereby forming a rope wrap 870. The rope wrap 870 includes taking a line of hair down from the top of the head down to a right ear region 840. The rope wrap 870 includes scooping up an assemblage of hair in front of the ear 830 and twisting it with the left hand 225, while the right hand 240 scoops up another assemblage of hair 830 towards a back of the head 850 and twists it into the twist 870. The right hand 240 continues to scoop up more hair assemblages 830 and twists it into the twist 870. The twisted temple-to-temple pull 820 continues scooping assemblages of hair into the twist, around the head, until the practitioner scoops as assemblage of hair on the other ear 860, on other side of head. Hair scooped earlier may fall from the assemblage as more scoops are performed, depending on the characteristics (length, etc.) of the hair. When there is no more hair to grab and twist in, then the hair may be dropped and the pull may be started over on the side opposite from the original starting side and going the opposite direction around the head.

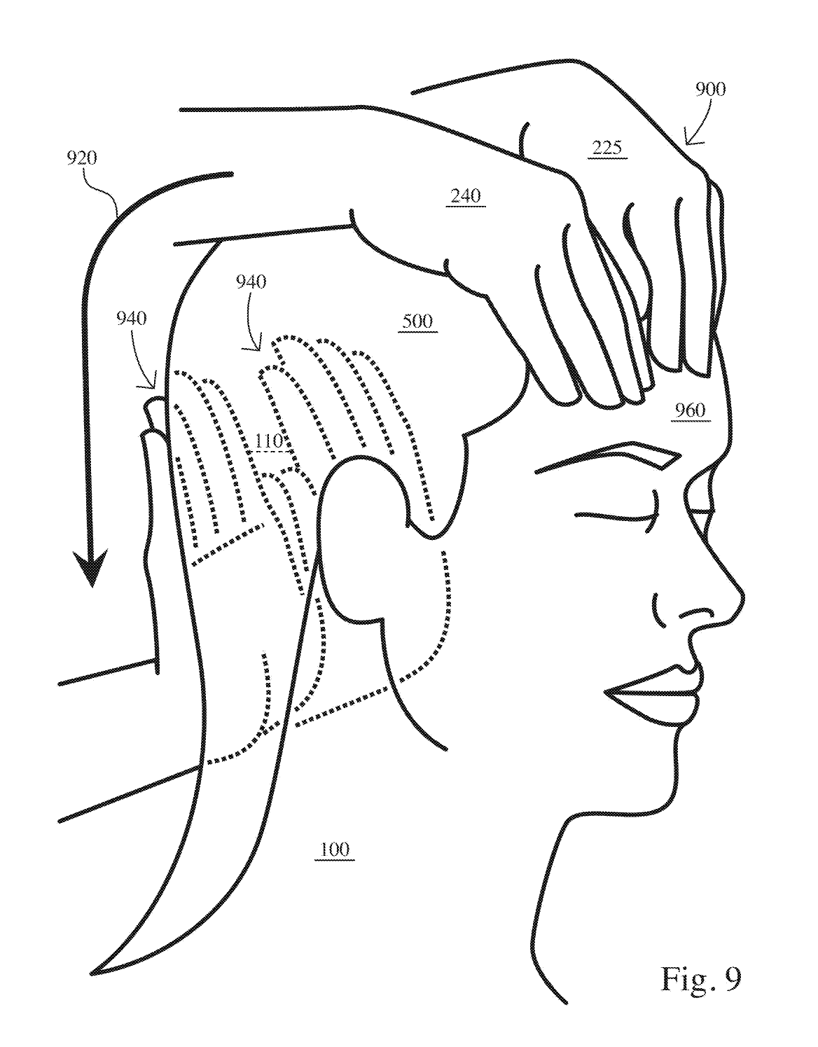

FIG. 9 is a side perspective view performance of a crown-to-nape pull, according to one embodiment of the invention. There is shown a left hand 225 and a right hand 240 of a practitioner, wherein the left and right hands are in a cupped-hand configuration 900 about a forehead region 960 of a head. The left and right hands are placed side-by-side about the forehead region 960 with the fingers placed above the eyebrows. The fingers and thumbs of each hand are closed and substantially parallel to the longitudinal orientation of the hair.

The illustrated technique may be referred to as a “palming technique” 900 configured to provide follicular muscle and nerve stimulation. The left and right hands 225, 240 are placed on the hair about a forehead region 960. In operation, the palming technique includes petting the forehead 960 back to a nape region 110, thereby resting the palm of each hand above a neck region 100, similar to petting a cat. The palming technique 900 includes putting the left and right hands 225, 240 on a top of the head 500 and pulling back and dragging 920 the hands 225, 240 across the top of the hair 940 ending substantially about the nape region 110. The palming technique 900 is a good technique to begin follicular muscle and nerve stimulation and to survey the overall lay of the hair, such as extensions, and etc. that could be pulled out by an implement.

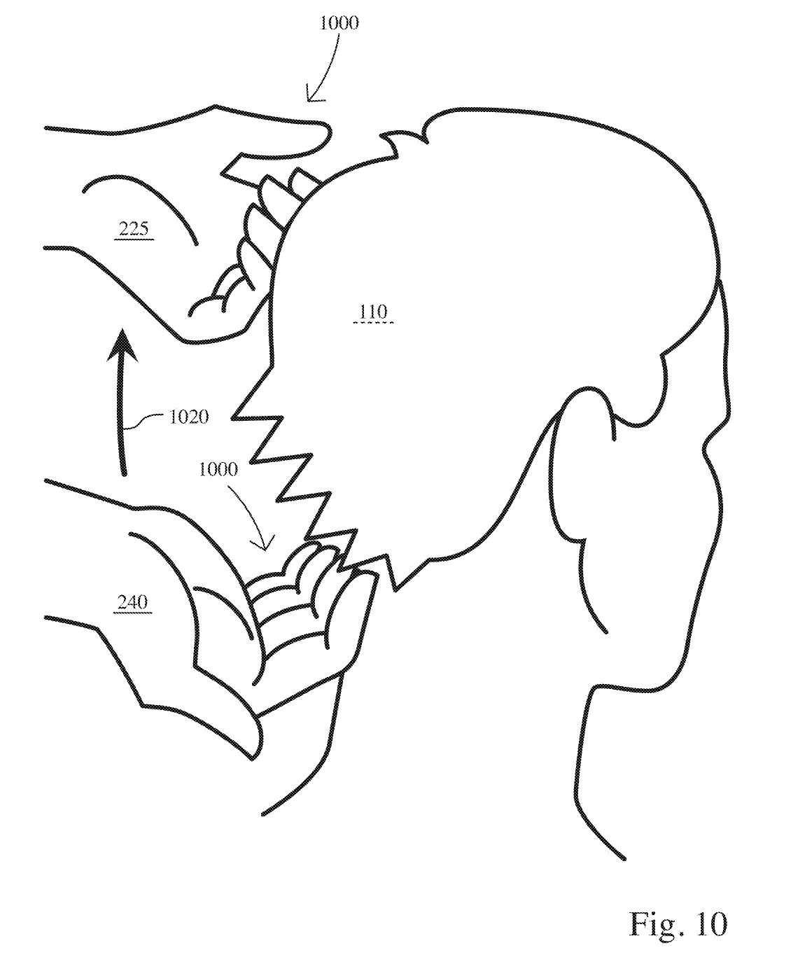

FIG. 10 is a back perspective view of performance of a transverse cascading nape-to-crown pull, according to one embodiment of the invention. There is shown a left hand 225 and a right hand 240 of a practitioner, wherein the left and right hands are configured in an open palm configuration 1000. The fingers of each hand are orientated substantially parallel to the longitudinal orientation of the hair. As illustrated, the method is configured to be performed on short hair about a nape region.

The illustrated technique may be referred to as a “rake” technique 1000 configured for follicular muscle and nerve stimulation of short hair. In operation, the rake technique 100 includes raking the fingers 1020, of a left hand 225 and a right hand 240, up against the grain of the hair. The rake technique 1000 also includes turning the hands over and raking with the grain of the hair and raking the hair from the front of the head to a nape region 110. The rake technique 1000 does not include trapping the hair between the fingers. Each hands 225, 240 alternates lifting the fingers through the hair, about a nape region, up towards the top of the head.

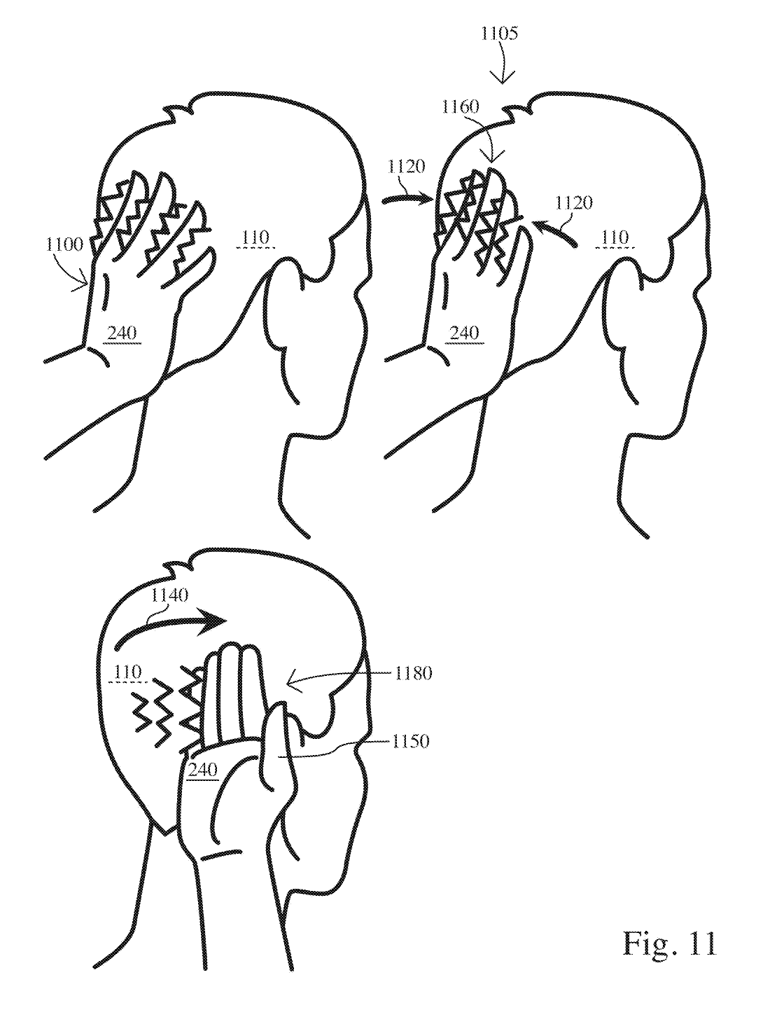

FIG. 11 is a back perspective view of a longitudinal hair pull, according to one embodiment of the invention. There is shown a right hand 240 of a practitioner, wherein the right hand 240 is configured in a flat palm configuration 1100. The open palm configuration 1100 includes placing the palm of the hand, fingers, and thumb flat against the hair, about a nape region 110 of the heir. The fingers of the right hand are orientated substantially parallel to the longitudinal orientation of the short hair. As illustrated, the practitioner is performing the hair pull on short hair.

The illustrated technique may be referred to as a “palm-over technique” 1105 and is configured to provide follicular muscle and nerve stimulation. In operation, the palm-over technique 1105 includes an open palm configuration 1100, placing a right hand 240 in a nape region 110 of the hair, and squeezing 1120 the fingers together to trap the hair assemblage, between the fingers 1160 and then rolling the right hand out 1140. The rolling includes a thumb 1150 pulling away from the hair assemblage, with the fingers still closed 1180, and the pivot point of the rolling is the side of the right hand 240, opposite of the thumb. The technique may include a palm-up technique, similar to the palm-over technique 1105, but the right hand 240 rolls off at the wrist first, instead of the thumb 1150, and the pivot point of the rolling is the fingertips with the palm positioned up. The technique may also include alternating each hand, and pulling the hair in alternating directions.

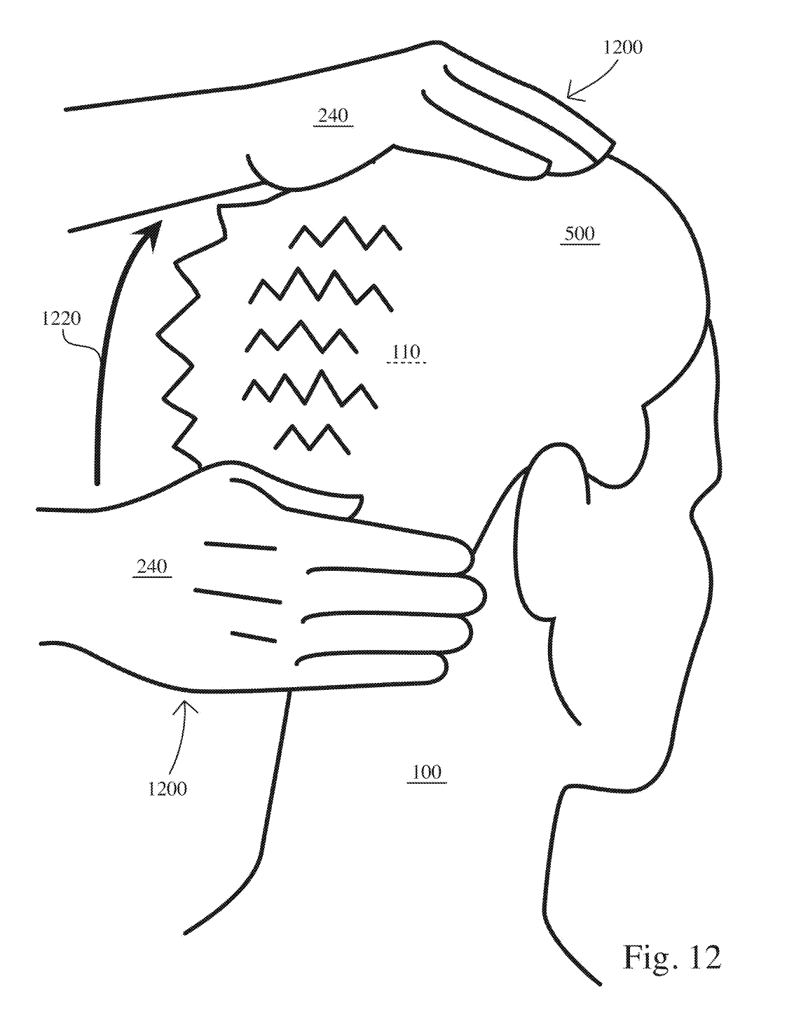

FIG. 12 is a back perspective view of performance of a nape-to-crown pull, according to one embodiment of the invention. There is shown a right hand 240 of a practitioner, wherein the right hand 240 is configured in a flat palm configuration 1200. The right hand 240 is placed above a neck region 100 with the fingers orientated substantially perpendicular to the longitudinal orientation of the hair, with the thumb facing up. As illustrated, the practitioner is performing the nape-to-crown pull on short hair.

The illustrated technique may be referred to as a “petting velvet technique” 1200 for short hair, such as the illustrated buzz haircut. In operation, the petting velvet technique 1200 includes cupping a right hand 240 to the shape of the head and rubbing the hair upward against grain/growth of the hair 1220. The right hand 240 begins rubbing the hair upwards from a neck region 100, through a nape region 110, up to a crown region 500 of the head. The petting velvet technique 1200 may include alternating each the left and right hands and trying not to touch the scalp of the head during the technique.

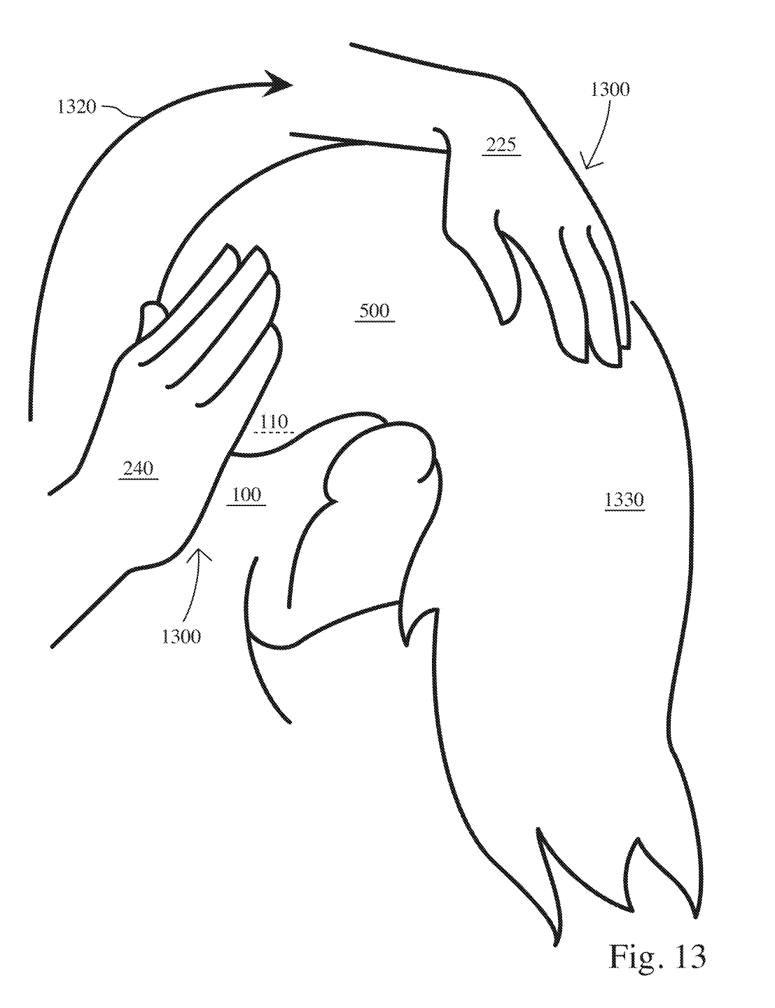

FIG. 13 is a back perspective view of nape-to-crown pull, according to one embodiment of the invention. There is shown a left hand 225 and a right hand 240 of a practitioner, wherein the left and right hand is in a closed palm configuration 1300 about a nape region 110 up towards a crown region 500 of the head. The fingers of the left and right hands are pressed against each other creating the closed palm configuration 1300. The fingers of the left and right hands are substantially orientated parallel to the longitudinal orientation of the hair.

The illustrated technique may be referred to as a “bottoms-up” technique 1320 configured to provide follicular muscle and nerve stimulation. The bottoms-up technique 1320 includes displacing the hair up and over the top of the head 500 and placing the palms of each hand 225, 240 on the hair, beginning about a neck region 100. The bottoms-up technique 1320 includes alternating each hand 225, 240 and dragging the hands along the hair, opposite of the grain of the hair. Each hand 225, 240 rubs the neck region 100, through a nape region 110, over the top of the head 500, and to the end of the hair 1330, applying pressure with the fingertips of each hand as the hands rub against the hair and head. The bottoms-up technique 1320 is similar to the petting velvet technique but the bottoms-up technique is configured to be performed on long hair. The bottoms-up technique 1320 includes cupping each hand 225, to the shape of the head and rubbing the hair upward against grain/growth of the hair 1300.

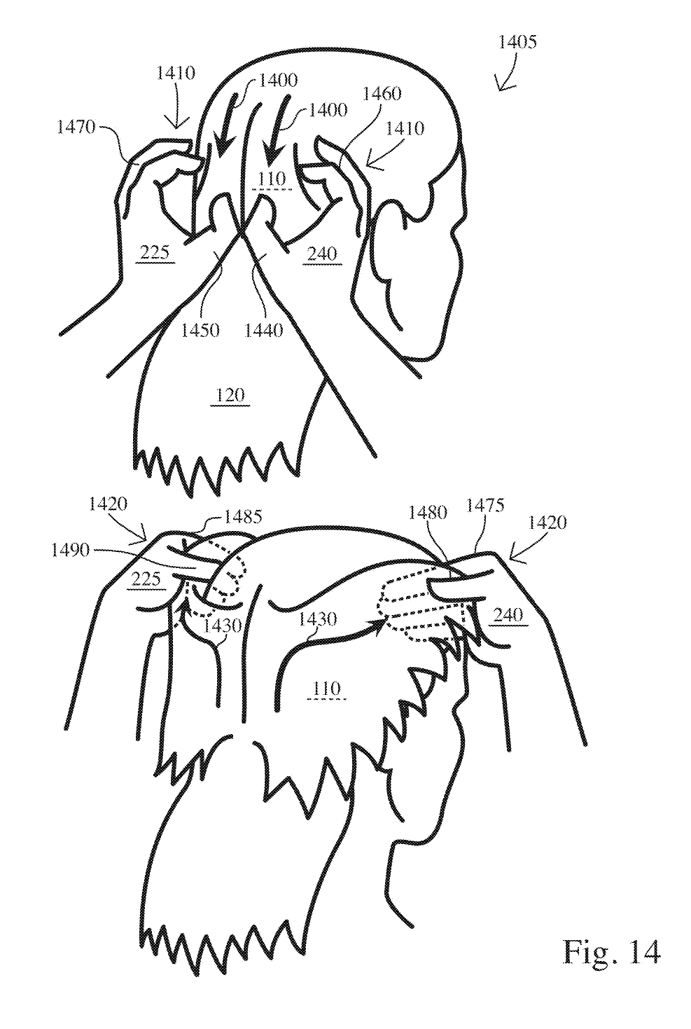

FIG. 14 is a back perspective view of a matched pair of transverse lateral-to-crown pulls, according to one embodiment of the invention. There is shown a left hand 225 and a right hand 240 of a practitioner, wherein the left and right hands are in a pinching configuration 1410. The thumb and forefinger of each hand is configured to pinch an assemblage of hair, wherein the fingers are substantially orientated parallel to the longitudinal orientation of the hair assemblage. The assemblage of hair is formed about a nape region of hair 110.

The illustrated technique may be referred to as an “angel technique” 1405 configured to provide follicular muscle and nerve stimulation. The angel technique 1405 includes using the forefingers 1460, 1470 and thumbs 1440, 1450 of each hand 225, 240; the technique 1405 includes parting the hair along the back of the head to make a pony tail with each hand 1400. The angel technique 1405 includes pinching 1410 at the bottom of each stroke and pulling the hands up and out 1430 from a nape region 110 of the head. Each hand 225, 240 is in a flat-duck handed configuration 1420, wherein the fingers 1475, 1485 and thumbs 1480, 1490 pull away from the nape region 110, thereby applying tension thereto, and releasing the hair thereafter. The technique 1405 includes pulling the hair assemblages up and out towards the front of the head, up and away from the part, and forward and out towards the ears. The angel technique 1405 is repeated around the back of the head in different areas.

In one embodiment of the invention, there is a method including performing successive longitudinal hair pulls include collecting a set of hair strands, thereby forming a hair strand assemblage. The successive longitudinal hair pulls also includes pulling the hair strand assemblage in a direction substantially aligned to the longitudinal direction of the hair strand assemblage.

In another embodiment of the invention, there is a thumb-in technique, a top-tail and back-tail drag techniques (and palm-over and rake for short hair) that tend to be performed first, because the techniques help relax the whole head and prepare the muscles and nerves for the treatment method. There is also a criss-cross technique, a rope technique, an angel technique, a pinch and pull technique, and a series of end techniques, such as a bottoms-up technique performed after the whole head techniques. These techniques are more stimulating and more intense and/or more intimate or allowing you to mess up their hair.

In one embodiment, a scanning technique is used, such as but not limited to a rake pull, wherein the scanning technique permits a practitioner to search the hair for tangles, extensions, and the like that may interfere with other pulls.

It is believed that pulls described herein, to varying degrees and advantageous effect, stimulate and/or otherwise affect the arrectores pilorum muscles, dermal nerves, and/or other systems associated with hair follicles to a degree that is in high contrast with associated epidermal and/or generalized and/or distributed dermal stimulation during such pulls. Accordingly, such affected systems are affected to a high degree in comparison to the surrounding tissue. It is believed that this isolated stimulation causes chemical and/or electrical changes in the brain and circulatory systems of humans that otherwise may not occur or may occur to a much smaller degree without such stimulation and/or during stimulation that is not in a similar high contrast. It is believed that such changes produce beneficial effects in the mood, receptiveness, intellectual and emotional patterns, and/or behaviors of most human recipients. It is believed that such effects occur in both genders and may perhaps be further pronounced and beneficial among human female recipients.

It is understood that the above-described embodiments are only illustrative of the application of the principles of the present invention. The present invention may be embodied in other specific forms without departing from its spirit or essential characteristics. The described embodiment is to be considered in all respects only as illustrative and not restrictive. The scope of the invention is, therefore, indicated by the appended claims rather than by the foregoing description. All changes which come within the meaning and range of equivalency of the claims are to be embraced within their scope.

For example, although the figures illustrate specific positions and orientations for the illustrated pulls, it is understood that such pulls may be performed using a near infinite variety of similar positions and/or orientations while still keeping to the characteristics of the pull. In fact, because heads and hands are of a great variety of size and shape among recipients and practitioners, the exact configurations are expected to be inifinite.

Additionally, although the figures illustrate performing pulls from behind a recipient, it is possible to perform most, if not all, of the illustrated pulls from other positions in relation to the recipient.

It is also envisioned that pulls may be performed in a near infinite variety of patterns with regard to what pulls are performed and in what order.

Pulls are illustrated with long and with short hair and it is understood that pulls may be adapted to the characteristics (length, friction coefficient, thickness, fragility, and the like) of a recipients hair.

It is expected that there could be numerous variations on the implementation of the invention. For example, any of the illustrated pulls may be performed with hands of a practitioner and/or with implements (brushes, clips, clamps, rakes, combs, and the like and variations and combinations thereof).

Finally, it is envisioned that the implements may be constructed of a variety of materials, including but not limited to plastics, rubbers, polymers, ceramics, fibers, woods, metals, composites, combinations thereof, and the like.

Thus, while the present invention has been fully described above with particularity and detail in connection with what is presently deemed to be the most practical and preferred embodiment of the invention, it will be apparent to those of ordinary skill in the art that numerous modifications, including, but not limited to, variations in size, materials, shape, form, function and manner of operation, assembly and use may be made, without departing from the principles and concepts of the invention as set forth in the claims. Further, it is contemplated that an embodiment may be limited to consist of or to consist essentially of one or more of the features, functions, structures, methods described herein.

What is claimed is:

1. A method of follicular muscle and nerve stimulation configured to generate a follicular stimulation effect in high contrast to epidermal stimulation, comprising the steps of:
a. performing a plurality of successive longitudinal hair pulls, wherein each of the longitudinal hair pulls includes:
i. collecting a set of hair strands, thereby forming a hair strand assemblage, and
ii. pulling the hair strand assemblage in a direction substantially aligned to the longitudinal orientation of the hair strand assemblage; and
b. performing a plurality of successive transverse cascading hair pulls, wherein each of the transverse cascading hair pulls includes:
i. collecting a set of hair strands, thereby forming a hair strand assemblage,
ii. pulling the hair strand assemblage in a direction substantially transverse to the longitudinal orientation of the hair strand assemblage, and
iii. releasing portions of the hair strand assemblage in a cascading manner as the hair is pulled.

2. The method of claim 1, further comprising performing a series of transverse cascading nape pulls.

3. The method of claim 1, further comprising performing a series of alternating transverse cascading lateral-to-crown pulls.

4. The method of claim 1, further comprising performing a series of transverse cascading crown pulls.

5. The method of claim 1, further comprising performing a series of longitudinal nape pulls.

6. The method of claim 1, further comprising performing a series of transverse cascading nape to crown pulls.

7. The method of claim 1, further comprising performing a series of crown pulls.

8. The method of claim 1, further comprising performing a twisted temple-to-temple pull.

9. The method of claim 1, further comprising performing a series of crown-to-nape pulls.

10. The method of claim 1, further comprising:
performing a pull selected from the group of pulls consisting of: transverse cascading nape pull, alternating transverse cascading lateral-to-crown pull, transverse cascading crown pull, longitudinal nape pull, transverse cascading nape to crown pull, crown pull, twisted temple-to-temple pull, and crown-to-nape pull; and
measuring a physiological characteristic of an individual receiving follicular nerve and muscle stimulation.

11. The method of claim 1, wherein step (a) is performed before performance of step (b).

12. The method of claim 1, wherein step (a) is repeated a plurality of times using differing pulls and then step (b) is subsequently performed a plurality of times.

13. A method of follicular muscle and nerve stimulation configured to generate a follicular stimulation effect in high contrast to epidermal stimulation, comprising the steps of:
a. performing a plurality of successive transverse cascading hair pulls, wherein each of the transverse cascading hair pulls includes:
i. collecting a set of hair strands, thereby forming a hair strand assemblage,
ii. pulling the hair strand assemblage in a direction substantially transverse to the longitudinal orientation of the hair strand assemblage, and
iii. releasing portions of the hair strand assemblage in a cascading manner as the hair is pulled.

14. The method of claim 13, further comprising performing a series of transverse cascading nape pulls.

15. The method of claim 14, further comprising performing a series of alternating transverse cascading lateral-to-crown pulls.

16. The method of claim 15, further comprising performing a series of transverse cascading crown pulls.

17. The method of claim 16, further comprising performing a series of longitudinal nape pulls.

18. The method of claim 17, further comprising performing a series of transverse cascading nape to crown pulls.

19. The method of claim 19, further comprising performing a twisted temple-to-temple pull.

20. A method of follicular muscle and nerve stimulation configured to generate a follicular stimulation effect in high contrast to epidermal stimulation, comprising the steps of:
a. performing a plurality of successive longitudinal hair pulls, wherein each of the longitudinal hair pulls includes:
i. collecting a set of hair strands, thereby forming a hair strand assemblage, and
ii. pulling the hair strand assemblage in a direction substantially aligned to the longitudinal orientation of the hair strand assemblage; and
b. performing a plurality of successive transverse cascading hair pulls, wherein each of the transverse cascading hair pulls includes:
i. collecting a set of hair strands, thereby forming a hair strand assemblage,
ii. pulling the hair strand assemblage in a direction substantially transverse to the longitudinal orientation of the hair strand assemblage, and
iii. releasing portions of the hair strand assemblage in a cascading manner as the hair is pulled.
c. performing a series of transverse cascading nape pulls;
d. performing a series of alternating transverse cascading lateral-to-crown pulls;
e. performing a series of crown pulls;
f. performing a series of twisted temple-to-temple pulls; and
g. measuring a physiological characteristic of an individual receiving follicular nerve and muscle stimulation using a measuring device.
